(19) United States
(12) Patent Application Publication
(10) Pub. No.: US 2022/0387693 A1
(43) Pub. Date: Dec. 8, 2022

(54) Device for the Extraction of Body Fluid by Suction

(71) Applicant: MEDELA HOLDING AG, Baar (SE)

(72) Inventors: Lukas Bannwart, Rotkreuz (SE); Ivo Ramella, Ebikon (SE)

(21) Appl. No.: 17/776,477

(22) PCT Filed: Nov. 11, 2020
(86) PCT No.: PCT/EP2020/081713

§ 371 Date: May 12, 2022

(30) Foreign Application Priority Data
Nov. 12, 2019 (EP) 19208627.0

(51) Int. Cl. A61M 1/00

(52) U.S. Cl. CPC A61M 1/65; A61M 1/60; A61M 1/86; A61M 2202/0014; A61M 2205/14; A61M 2205/6018

(57) ABSTRACT

The present invention relates to a device for the extraction of body fluid by suction, comprising a pump (2) with a pump housing (4) that comprises a suction opening (31), a container (6) with a container housing (8) that surrounds a reservoir (10) for storing the extracted body fluid and comprises an interface (84) in a container housing wall (82) for the communication between the suction opening (31) and the reservoir (10), a suction line port (74) that is connectable to a suction line (6) for the extraction of body fluid by suction, which is provided with a cover element covering the coupling device to improve the cleanliness and/or for the formation of functional surfaces for securing the suction line and/or releasing the coupling device.

The present invention relates to a device for the extraction of body fluid by suction. The device according to the invention has a pump with a pump housing which comprises a suction opening. In addition, a container with a container housing which surrounds a reservoir for storing the extracted fluid is provided. Provided in a container housing wall is an interface for the communication between the suction opening and the reservoir. Furthermore, a suction line port for the extraction of body fluid by suction is provided, where this suction line port is connectable to a suction line and in communicates with the reservoir.

In the device, the container is detachably connected to the pump housing. A coupling device is provided for this purpose. Such a device is known, for example, from EP 2 895 213 B1.

A device for the extraction of body fluid by suction is usually used in medical care. Sometimes they are used under sterile conditions. It is then to be ensured that the device is easy to clean and, if necessary, to disinfect. The outer surfaces of the device should therefore be easy to clean, requiring a smooth surface that is free of undercuts. In particular, the region of the coupling device, which can be formed, for example, by a locking projection in the form of an engagement hook interacting with an engagement tab, represents a region in which contamination can settle in.

The device is used over a long period of time, for example, in the context of wound treatment by vacuum therapy which is described in EP 2 525 866 B1. In such applications, care must be taken in particular to ensure that the suction line is reliably and permanently connected.

The present invention has the object of providing a solution that is improved with regard to at least one of the above requirements.

To satisfy this object, the present invention proposes a device having the features of claim 1.

A cover element covering the coupling device is provided in this device. Edges and/or undercuts of the coupling device on the outer side of the pump housing or the container housing are thereby typically covered. The coupling device is disposed beneath the cover element. The cover element is a member which is produced as a component separate from the pump housing and the container housing. The cover element is connected to at least one of the two housings as a separate component.

With the usual setup, the coupling device is located in a region of the two that in the operating position of the pump housing and the container housing is at the top. The coupling device typically comprises locking or engagement elements interacting with each other. For detachably coupling the container housing to the pump housing, it is usually necessary that a pawl projects from one of the containers and has an upwardly extending engagement projection which interacts with a downward-facing engagement tab that is provided on the other one of the housings. The pawl must necessarily be disposed lower, at least in sections, whereby a depression must be provided in the region of the coupling device, in particular since the pawl should be able to pivot downwardly to release the engagement connection. At least this region of the coupling device is preferably covered by the cover element.

The cover element is preferably made of plastic material. The same applies to the pump housing and/or the container housing. Since the container is typically a disposable product, the container housing should be as inexpensive as possible. The container housing can be formed integrally by way of blow-molding. The previously mentioned suction line port is typically formed preferably integrally on the container housing. The container can also be formed having multiple parts. It can in particular have a relatively non-specifically formed canister which is formed by way of blow-molding or injection-molding and is connected to a pan by adhesive bonding or welding, in particular ultrasonic welding, which can be produced by injection-molding and can form special functional surfaces, openings or functional elements of the container, such as the previously mentioned suction line port or a filter or check valves, which can prevent the undesirable ingress of extracted body fluid from the reservoir towards the suction opening. The pan there also preferably forms the container housing wall with the interface for the communication between the suction opening and the reservoir, where the previously mentioned valve or a filter, respectively, can be directly associated with this interface on the container housing side.

The pan preferably forms all functional surfaces for detachably coupling the container housing to the pump housing. The pan further preferably forms the suction line port and possibly further ports for further lines of the device.

The cover element can be connected to the pump housing. However, it is preferably connected to the container housing. The connection can be a positive-fit and/or force-fit and/or positive substance-fit connection. The cover element can be, in particular, adhesively bonded, welded and/or locked to the associated housing. However, ways of attachment which meet the requirement of economic production of the container housing and the cover element are to be preferred.

The cover element is preferably associated with the container housing and they are discarded together. With regard to the simple assembly of the container housing and the cover element, it is proposed according to a preferred further development of the present invention to connect the cover element in a positive-fit and/or force-fit and/or positive substance-fit manner, in particular by way of a locking mechanism, to the container housing. This locking mechanism is a positive-fit mechanism. The cover element is preferably latched to the container housing with the locking mechanism.

This type of connection between the cover element and the container housing is chosen in particular with regard to the preference that the container housing be preferably completed and put onto the market by the manufacturer together with the suction line connected to the suction line port. The three previously mentioned parts, namely the container housing, the cover element, and the suction line can then first be manufactured separately and joined in a simple manner by locking the cover element to the container housing and mounting the suction line onto the suction line port. The suction line port is typically a nozzle onto which the suction line is pushed while radial stretching a tube typically formed on the suction line, so that the suction line is already sufficiently connected to the suction line port. The suction line port is preferably covered by the cover element, so that this projection is covered by the cover element in the mounted state of the device, and a hygienically sound solution is offered there as well. Where the suction line port and the coupling device are typically disposed in the longitudinal direction of the container housing or the pump housing at the same height. The longitudinal direction is the direction in which the pump housing and the container housing are provided one behind the other. One direction transvers thereto extending vertically is the height direction. The other orthogonal, extending transverse to the longitudinal direction, is referred to as the width direction. The cover element typically extends in the width direction over almost the entire width of the container housing. In the region of the coupling between the container housing and the pump housing, it can be slightly reduced in width as compared to the usual width of the container housing. The container housing typically has the same width as the pump housing. The pump housing in the region of the coupling device preferably forms faces which enclose between themselves the region of the pump housing that is reduced in width and that can be preferably formed by the pan. The coupling device is disposed between these two faces and is accordingly covered at the side by the faces. The pump housing typically transitions in a flush manner to the container housing. At least the surface and the side surfaces of the device also exhibit no projections at the phase boundary between the pump housing and the container housing, so that the device can be easily cleaned and looks appealing also after the installation of the container housing on the pump housing.

If the coupling device is formed by interacting engagement elements, where the pawl can be formed on the container housing or the pump housing or the cover element. The pawl is preferably formed integrally on the pump housing, the container housing or the cover element, respectively, where the respective components in this case are preferably formed from plastic material.

If the pawl is formed by the cover element itself, then the engagement projection does typically project in the longitudinal direction over a cover surface formed by the cover element which is typically provided at the same height as the surface of the pump housing and the container housing. A design fulfilling the hygienic requirements is also obtained in that the engagement tab interacting with the engagement projection is formed by the pump housing, namely in such a way that the engagement projection in the locked state is located below the surface of the container housing. The surface of the container housing can then reach up to the surface of the cover.

If the pawl is formed by the container housing, then it can interact in the same way with an engagement tab of the pump housing. The separately provided cover element can be formed in the best possible manner to cover this coupling device. The cover element preferably forms a push button interacting with the coupling device, with the manual actuation of which the container housing can be decoupled from the pump housing. This push button interacts with the pawl. However, the push button provided on the cover element can also be part of the pawl which protrudes from the push button in the longitudinal direction and interacts with an engagement tab provided the side of the pump housing. The same arises where the pawl is provided integrally on the pump housing and interacts with an engagement tab which is formed by the cover element or the container housing. In the first case, the cover element is at least in part movable elastically relative to the container housing. In the second alternative, the cover element is typically provided rigidly relative to the container housing, whereas the push button is provided movable but integrally formed on the pump housing.

The cover element of the device according to the invention can preferably also be used to attach the suction line to the container with improved pull-out force. For this purpose, the cover element comprises a passage opening for the suction line and forms a securing surface which is typically associated with the passage opening. The securing surface is there formed in such a way that its abutment against the suction line causes a clamping force acting upon the suction line against the suction line port. Accordingly, the suction line is clamped between the securing surface and the circumferential surface of the suction line port. The securing surface can there in part or entirely surround the suction line port. After attaching the cover element to the container housing, the suction line is accordingly held with increased pull-out force on the suction line port.

If a cover element thus configured also forms the push button, then the push button is typically located in the width direction at the center on the cover element, whereas the at least one securing surface is provided at the side of the cover element. Corresponding to this, the connection to the suction line or a connection for a further line, for example, associated with the container, is usually provided off-center, generally close to the edge.

The cover element, for the best possible mounting of the push button preferably attached integrally and pivotably thereto, forms two mounting segments which accommodate the push button therebetween and are connected to the container housing. Engagement with the container housing is effected preferably only by way of these mounting segments. These mounting segments also preferably represent the previously mentioned securing surface for an associated line. The push button can be connected to the mounting segments by way of an integral hinge. However, configurations are also conceivable where the push button is provided with a spring arm which gives the push button the desired movability for the interaction with the pawl.

In addition to the above-mentioned suction line, a ventilation line which is typically in communication with and connected to a ventilation valve provided in the pump housing can also be provided. The ventilation valve can be actuated by controlling the pump. When using the device according to the invention as a suction pump for vacuum therapy, the ventilation line leads to the wound region that is covered in a substantially airtight manner. By opening the ventilation line, additional air can be introduced into this region, whereby the flow rate in the suction line is increased and it is flushed.

A ventilation line port is provided for the ventilation line on the side of the container and is in communication with a ventilation duct that leads into the pump housing. If the container housing has multiple parts, then the ventilation duct is typically formed by the pan that is formed in a more complex manner. Also for this ventilation line port, the termination element preferably forms a securing surface through which the ventilation line is mounted to the device with the increased pull-out force. If the cover element is connected to the container housing from the outset, then the former can basically connect the respective lines captively to the container housing, so that it can be delivered as a unit to the user.

The device according to the invention is preferably further developed by an intermediate module which is provided with a feed pump and comprises inlet and outlet openings for a feed hose interacting with the feed pump. The feed pump can be a roller pump that acts directly upon the feed hose to deliver the medium disposed in the feed hose. This medium can be a pharmaceutical fluid. The feed pump can have a data connection to a control device accommodated in the pump housing for controlling the pump for the extraction of the body fluid by suction and can be controlled thereby. Alternatively, the feed pump can also have a control device that is formed and arranged within the intermediate module. In the first case, the intermediate module typically has interfaces in the form of touch surfaces which establish contact with corresponding interfaces formed on the pump housing side when the intermediate module is coupled to the pump housing, so that the module has data connection to the pump housing. The same applies to contacts of a possible power supply of the intermediate module via the power supply of the pump housing.

The intermediate module is detachably connected by way of coupling devices to the container housing and the pump housing. The intermediate module is there typically formed such that it comprises coupling elements corresponding to the locking or engagement elements of the coupling device between the pump housing and the container housing. If the container housing is formed with a pawl as the locking projection, then the intermediate module corresponding thereto comprises an engagement tab interacting with the pawl, whereas on the opposite side, the intermediate module comprises a pawl interacting with the engagement tab of the container housing. If the container housing has the locking projection for coupling to the pump housing, then the intermediate module comprises oppositely disposed therefrom the engagement tab interacting therewith, and oppositely disposed therefrom the locking projection which interacts with the associated engagement tab of the container housing.

Also any other elements for mechanically coupling are provided on the intermediate module corresponding to the container housing and the pump housing. The intermediate module preferably forms a through hole which forms a section of a line for the suction air between the reservoir and the pump The intermediate module can be inserted between the container housing and the pump housing, while the function of the elements of the coupling device which typically couple the container housing to the pump housing are maintained. The previously mentioned cover element covers at least one of the coupling devices between the intermediate module and the container housing or the pump housing.

A second cover element is preferably provided and covers the other of the coupling devices of the intermediate module. This second cover element preferably comprises at least one passage opening for the feed hose. The second cover element is accordingly specifically adapted to the requirements of the intermediate module.

According to one preferred further development of the present invention, the pump housing has at least one mounting element. The container housing comprises at least one mounting mating element interacting with the mounting element. Due to this interaction, the container housing is mounted pivotable relative to the pump housing. This mounting is typically effected close to the base, so that, for detachably coupling the container housing, the latter is first pre-positioned relative to the pump housing by way of the mounting element. The mounting element and the mounting mating element are formed such that they allow a pivoting motion of the container housing relative to the pump housing such that the coupling device securing the connection between the container housing and the pump housing is activated at the end of a pivoting motion of the typically upper end of the container housing.

The mounting element is preferably located within the previously mentioned pans of the pump housing. A mounting element, preferably in the shape of a pin or projection, is provided preferably on the inner side of the oppositely disposed pans. Corresponding to this, the container housing, preferably its pan, comprises oppositely disposed thereto the mounting mating element in the form of a recess formed on the container housing, into which the pin or projection engages. The free space formed between the mounting elements can then also be used as a reservoir. The mounting element or the mounting mating element, respectively, are preferably formed integrally in the form of segments or functional surfaces on the container housing or the pump housing, respectively.

The intermediate module is also preferably formed in the manner described above, where it forms a mounting element on one side and a mounting mating element on the opposite side according to the associated elements of pump housing and container housing. The intermediate module, like the container housing, can be detachably attached to the pump housing and the container housing can be detachably attached to the intermediate module as well as to the pump housing.

According to a preferred further development of the present invention, a detection device is typically provided on the pump housing or within the same and is adapted to detect an identifier which is provided on the container housing and/or the intermediate module. The detection device has a data coupling to the motor control device of the pump via a signal line preferably provided within the pump housing. It can then be communicated by way of the identifier to the motor control device which element is provided. The identifier can also be used to detect authorized consumables and to block the operation of the pump in the event of unauthorized consumables. Also, correct positioning of the container housing relative to the pump housing can be verified by the detection device and the associated identifier, as is known for example from EP 0 853 950 B1. The identifier can be formed by a transponder attached to the intermediate module or the container.

A container for storing extracted body fluid is also claimed with the present invention as an essential part of the inventive. It comprises a container housing which surrounds a reservoir for body fluid. The features and further developments already previously discussed in the context of the device as such apply for this container housing. The container has an interface that is connectable to a suction opening of a pump housing and leads to the reservoir. The container also has a suction line port that is in communication with the reservoir which is connected to that of a suction line for the extraction of fluid by suction. The configurations and further developments discussed above apply to the suction line port and the suction line. The container has a cover element with at least one passage opening for the suction line and at least one securing surface due to the abutment of which against the suction line a clamping force is effected in the axial direction clamping the suction line against the associated suction line port. This cover element is preferably formed as described above.

The independent aspect of the present invention specifies an entity of a container, a suction line and a cover element, where the suction line is attached with an increased pull-out force to the container housing due to the interaction of the cover element and suction line port.

Further details and advantages of the present invention shall become apparent from the following description of embodiments in combination with the drawing, in which:

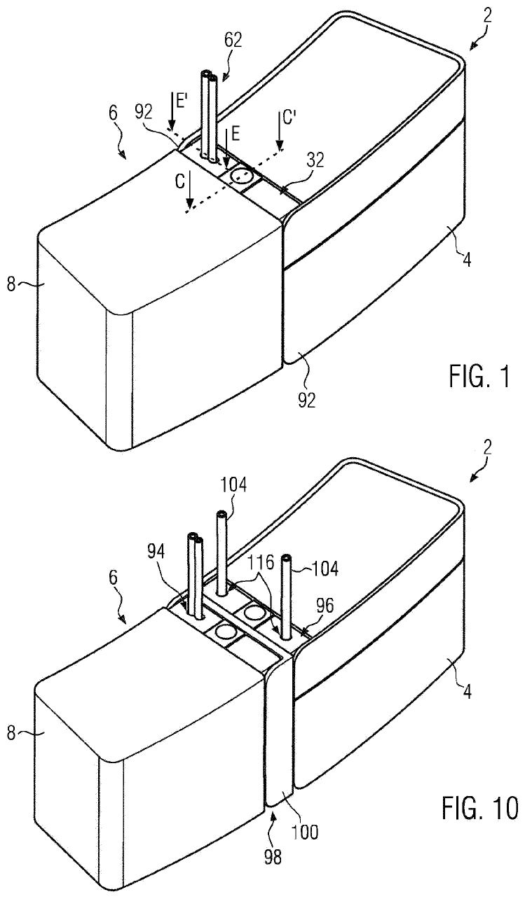

FIG. 1 shows a perspective side view of an embodiment of a device for the extraction of body fluid by suction;

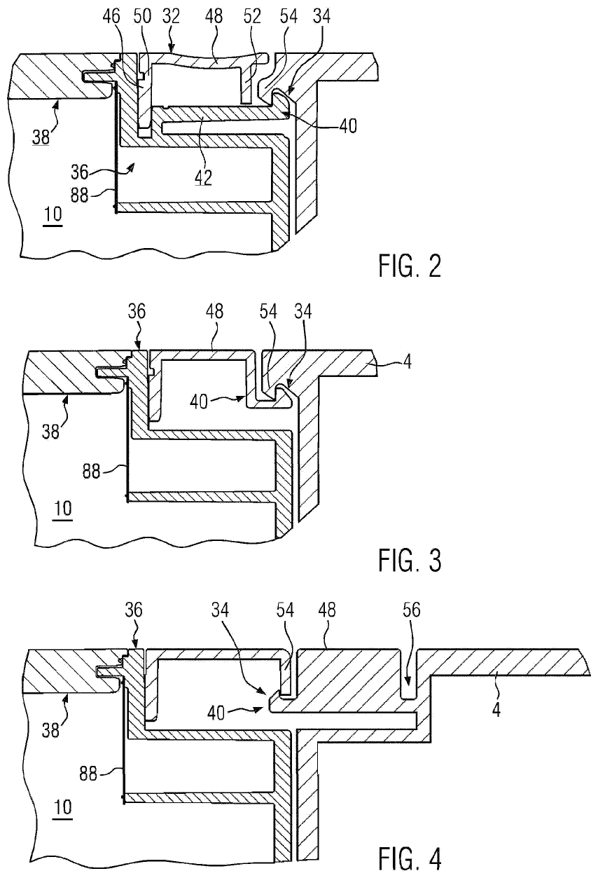

FIG. 2 shows a sectional view along the C, C′ according to FIG. 1 for a first variant according to FIG. 1 for a first variant of a coupling device;

FIG. 3 shows the sectional view according to FIG. 2 for a second variant of a coupling device;

FIG. 4 shows the sectional view according to FIGS. 2 and 3 for a third variant of a coupling device;

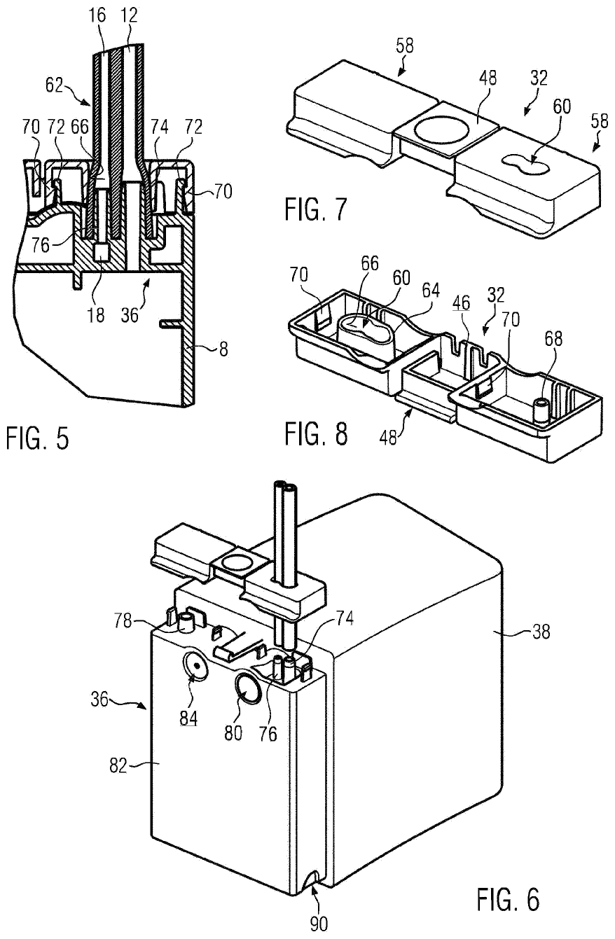

FIG. 5 shows a sectional view along line E, E′ according to FIG. 1;

FIG. 6 shows a perspective side view of the container of the embodiment with the cover element and various lines in an explosion representation;

FIG. 7 shows a perspective side view on the upper side of the cover element of the embodiment;

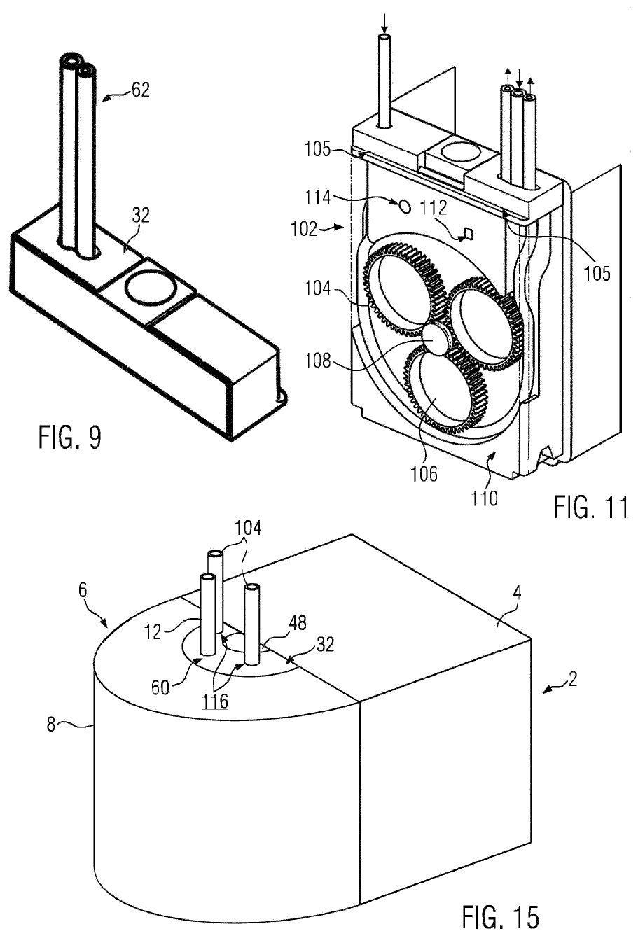

FIG. 8 shows a perspective lower view of the cover element of the embodiment;

FIG. 9 shows a rear top view onto a variant of the cover element;

FIG. 10 shows the embodiment according to FIG. 1 with an intermediate module arranged between the pump housing and the container housing;

FIG. 11 shows a perspective top view that reveals the details of the intermediate module;

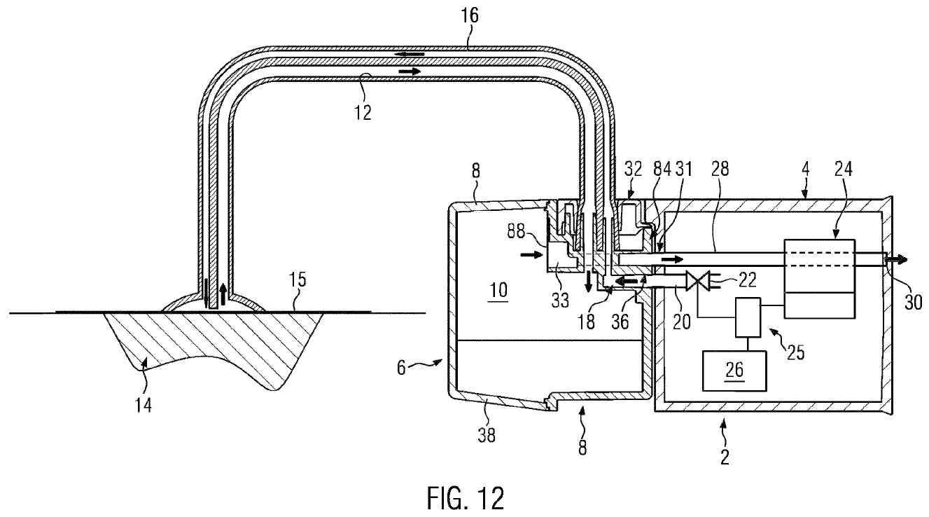

FIG. 12 shows a schematic diagram for connecting the device for the extraction by suction in the framework of vacuum therapy;

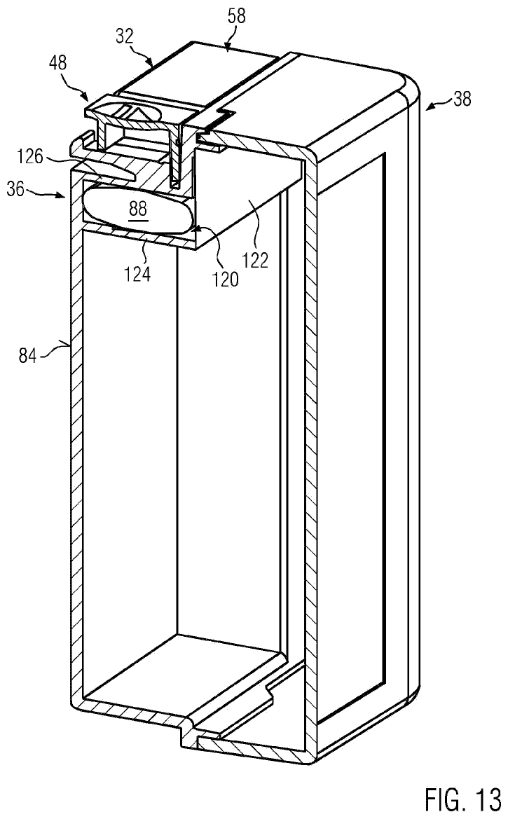

FIG. 13 shows a perspective cross sectional view of an alternate embodiment of a container;

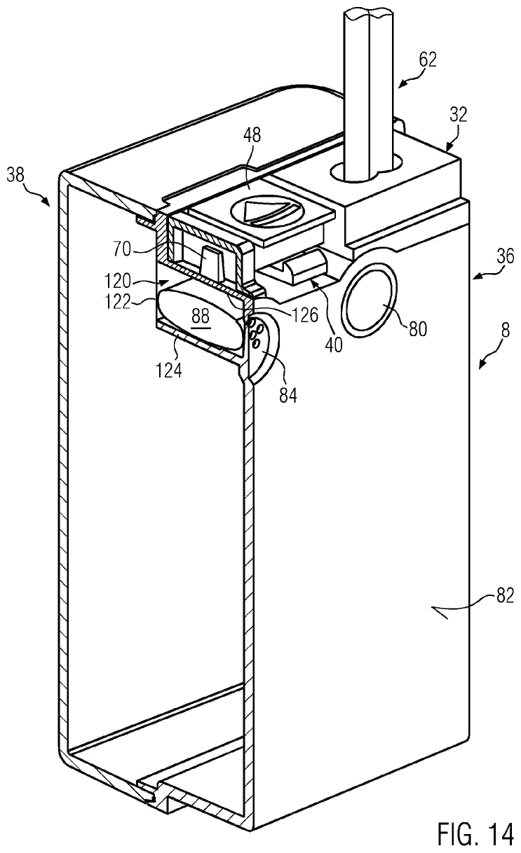

FIG. 14 shows a perspective cross sectional view opposite to the view of FIG. 13 for the embodiment of the container of FIG. 13 and

FIG. 15 shows a perspective side view of an alternative embodiment of a device for the extraction of body fluid by suction.

FIG. 1 shows a perspective side view of an embodiment with a pump 2 comprising a pump housing 4 that defines the outer surfaces of the pump 2. Detachably connected to this pump 2 is a container 6 whose outer surfaces are formed by a container housing 8. The container housing 8 forms a reservoir 10 (cf. FIG. 12) in which body fluid to be extracted by suction is collected. For this purpose, the embodiment comprises a suction line 12 that leads to the reservoir 10 and can be connected to a wound region 14 which is provided with a dressing over which a substantially airtight film 15 is applied that is typically adhesively connected to the skin surrounding the wound, so that the wound region 14 can communicate with the environment only via lines connected thereto. In addition to the suction line 12, a ventilation line 16 is provided according to FIG. 12 which leads to the container 6 and leads via a ventilation duct 18 that is in the container 6 and formed integrally thereon with an inner ventilation line 20 within the pump 2 to a ventilation valve 22.

FIG. 12 further illustrates a suction pump 24, which is provided in the pump housing 4 and associated with a control device 25, which also has a data coupling to the ventilation valve 22. The pump 2 is provided with an autonomous power supply in the form of accumulator batteries 26. The suction pump 24 is in communication with the reservoir 10 via an inner suction line 28 within the pump housing 4 and comprises an exhaust 30 leading to the exterior for the air ejected from the suction pump 24 during operation.

The illustration according to FIG. 12 is only schematic. Disposed between the inner suction line 28 and the reservoir 10 can be a filter which clogs when wetted with moisture to prevent the fluid extracted by suction from being sucked into the pump 2. The exhaust 30 can be provided with silencing elements and/or a filter.

The free end of the inner suction line 28 is formed by a suction opening 31 which is formed on an outer surface of the pump housing. The inner suction line 28 is in communication via this suction opening 31 with a suction duct 33 formed within the container housing 6 and integrally thereby. When the container housing 6 is attached to the pump housing 4, the suction duct 33 and the inner suction line 28 or the inner ventilation line 20 and the ventilation duct 18, respectively, are connected in a sealed manner by way of surfaces of the pump housing 4 or the container housing 8 that bear against each other.

A cover element 32 can be seen in FIG. 1 between the pump 2 and the container 6 and shall be explained in more detail below. The cover element 32 has a flush surface with the surfaces of the pump 2 and the container 6. The surfaces transition into each other in a flush manner. The cover element 32 is made of plastic materials by injection molding and is formed as a separate component and locked by a locking mechanism to the container housing 8.

The cover element 32 is used to cover a coupling device 34 which couples the container housing 8 detachably to the pump housing 4. Formed in the embodiment shown in FIG. 2 for this purpose on a pan 36, which together with a canister 38 forms the container housing 8 and surrounds the reservoir 10, is a locking projection 40 formed as pawls which is pivotable relative to the pan 36, for which purpose presently an end of the pawl 42 on the attachment side is tapered in cross-section, whereby a kind of integral hinge is formed. Provided between the end of the pawl 42 on the attachment side and a connecting surface of the pan 36 welded to the canister is a reception slot 44 which receives and positions the free end of a rear wall 46 of the cover element 32.

The sectional view shown in FIG. 2 through the cover element 32 intersects a push button 48 which is connected only via the rear wall 46, namely via an integral hinge formed by a tapered cross section 50. Projecting from the underside of the push button 48 is an actuation web 52 which in its locked position shown in FIG. 2 upon actuation of the push button 48 against the locking projection 40 interacts with an engagement tab 54 formed by the pump housing 4. The coupling device 34 can thus be decoupled by actuating the push button 48.

In the embodiment according to FIG. 3, the locking projection 40 is provided at a free end of the push button 48 and projects thereover. The engagement tab 54, as a further element of the coupling device 34, is formed like previously by the pump housing 4.

In the third variant shown by way of example, the push button 48 is formed integrally on the pump housing 4 and mounted via a hinge 56 elastically pivotable relative thereto. Formed at the free end of the push button 48 is the locking projection 40 which engages behind the engagement tab 54 formed by the cover element 32. The pump housing 4 and the container housing 8 are also detachably coupled to each other by this embodiment.

As illustrated by FIGS. 7 and 8, the push button 48 is provided in the width direction at the center on the cover element 32. It is located between two mounting segments 58, of which the mounting segment 58 in FIG. 7 at the right forms a passage opening 60 for a tube 62 which forms the suction line 12 and the ventilation line 16 in combination (cf. FIG. 5). The passage opening 60 is formed and circumferentially surrounded by a clamping cylinder 64 which forms a securing surface 66. The other mounting segment similarly has a positioning cone 68 projecting over itself. The two mounting segments 58 are substantially pan-shaped. The push button 48 is cut free relative to the surfaces of the mounting segments 58 and is only connected by way of the rear wall 46 to the other components of the cover element 32. Projecting from the inner surfaces of the pan-shaped mounting segments 58 are locking projections 70 which interact with engagement heads 72 formed on the container housing 8, specifically on the pan 36, and form a locking mechanism with which the cover element 32 is connected to the container housing 8 in a positive-fit manner.

This connection can be gathered in particular from FIG. 5 which also illustrates the attachment of the tube 62 to the container housing 8. For this purpose, the latter has a suction line port nozzle 74 and a ventilation line port nozzle 76 which are formed integrally by the pan 36 and project upwardly from the latter (cf. FIG. 6). The material of the tube is clamped between these port nozzles 74, 76 and the securing surface 66. This secures the tube 62 on the container housing 8 with increased pull-out force. In addition, the port nozzles 74, 76 also act as positioning aids in the framework of the assembly of the cover element 32. The same applies to the interaction of the positioning cone 68 with a cone seat 78 which is provided for the oppositely disposed mounting segment 58 on the housing side (see FIG. 6).

As conveyed by FIGS. 5 and 6, the ventilation line 16 is in communication with the ventilation duct 18 via the ventilation line port nozzle 76 within the pan 36. The ventilation duct 18 terminates at a ventilation interface 80 which is exposed at a face side container housing wall 82 of the container 6 and which in the mounted state is in communication with the ventilation duct 20 within the pump housing 4 (see also FIG. 12). In addition to the ventilation duct 18, a further opening as an interface 84 can be seen on the container housing wall 82 and is in communication with the reservoir 10 via the suction duct 33 within the pan 36. The inlet of this suction duct 33 is formed through a rear wall of the pan 36 which is provided with a filter 88 which covers the access on the reservoir side to the suction duct 33 (cf. FIGS. 2-4, 12). For reasons of simpler illustration, the ventilation duct 18 and the suction duct 33 in FIG. 12 are drawn on top of each other and not side by side. Either configuration is conceivable for the realization of the invention.

The combination of FIGS. 5 and 12 illustrates that the flow paths within the container 6 are formed solely by the pan 36 which can be produced as a complex plastic component by way of injection molding. The canister 38, in comparison, is a non-specifically formed hollow body which only needs to form adapted joining surfaces for being joined with the pan 36.

FIG. 6 at the lower end of the pan 36 shows a mounting pin mating element 90 which is formed as a semicircular cavity. The oppositely disposed face surfaces of the pan 36 each have a corresponding cavity. The latter is first fitted onto mounting elements, not shown, corresponding thereto and formed in the pump housing 4 when the container 6 is attached. This creates a pivot mount about which the container 6 is pivoted when coupled to the pump 2 until the coupling device 34 engages. For this purpose, the pump housing 4 has lateral faces 92 from which the mounting elements project and which form a receptacle for the pan 36, offset inwardly relative to the remaining housing of the container, together with the cover element 32 (cf. FIG. 1).

The essential elements and advantages of the basic form of the invention have now been illustrated. A tube 62 can be connected captively to the container 6 via the cover element 32. The cover element 32 and the container 6 are first manufactured separately and locked to each other with the inclusion of the tube 62. The cover element 32 also covers parts of the coupling device 34, so that an aesthetically appealing image on the outer side arises. The device is easy to clean and to keep clean. The container 6 is formed in two parts, where solely the pan 36 embodies with more complex functional surfaces for locking the cover element 32 and/or for flow guidance of extracted body fluid or ventilation air. This part of the container 6 also forms interfaces to the ducts on the pump side for the extraction of body fluid by suction and the ventilation of this flow path. These interfaces are each sealed. The pan 36 is also provided with the filter 88.

The pump housing 4 forms a suitable receptacle for the accommodation of the pan 36, so that also the side surfaces of the pump housing 2 and the container housing 8 transition to each other in a flush manner. The pump housing 4 is joined with the container housing 8 by a pivot motion. The two housings 4, 8 are necessarily locked to each other by the coupling device 34 at the end of this pivot motion.

FIG. 10 illustrates the previously discussed embodiment in which an intermediate module 94 is arranged between the pump 2 and the container 6. The intermediate module 94 has mounting elements and mounting mating elements on opposite sides, so that the intermediate module 94 can be connected to the pump housing 4 like the container 6 and the container 6 can be connected to the intermediate module 94 like to the pump housing 4. The intermediate module 94 also has the previously described elements of the locking mechanism for attaching another, second cover element 96 to the intermediate module 94. A correspondingly configured intermediate module housing 98 fits in the side surfaces and the surface of the device in a flush manner, so that this variant also fulfills the hygienic requirements. Accordingly, the intermediate module 94 is detachably connected by way of a coupling device to the pump housing 4, and by way of a further coupling device detachably to the container housing 8. The respective coupling devices are covered by the cover element 32 and the second cover element 96.

The intermediate module housing 98 accommodates a pump 102 which is formed as a peristaltic pump and comprises planetary wheels 106 that interact with a feed hose 104 and that move around a driven shaft 108 during operation. The intermediate module 98 forms inlet and outlet openings 105 for this feed hose 104 by way of which the feed hose is led into the intermediate module. A face-side housing wall 110 of the intermediate module housing 98 comprises two passage openings, one of which form a ventilation duct segment 112, and the other a suction duct segment 114. The ventilation duct segment 112 connects the ventilation duct 18 to the internal ventilation line 20 within the pump 2. The suction duct segment 114 connects the interface 84 with the internal suction line 28 on the pump side.

FIGS. 13 and 14 show an alternate embodiment of a container. The cover element 32 corresponds with the embodiment depicted in FIG. 2. The major difference between the embodiment of FIG. 2 resides in the alternate realization of the filter. In the embodiment of FIGS. 13 and 14, the filter 88 is received within a receptacle 120 formed between a membrane 122 and the container housing wall 82, which receptacle is defined between a peg 124 and an upper wall section 126 of the container housing 8. The membrane 122 per se can have the function of a filter. In addition, the filter 88 in this embodiment is an activated carbon filter consisting of an outer envelope containing the activated carbon. This activated carbon filter may be pressed into the receptacle 122 such that air sucked from the reservoir 10 into the inner suction line 28 inevitably needs to pass the filter 88.

FIG. 15 shows a perspective side view of an alternative embodiment. Same components are marked with the same reference numerals as in the embodiment previously described. The cover element 32 is presently formed to be semicircular. The push button 48 is located at the center of the cover element 32. The cover element 32 forms different openings for passing lines in a ring-shaped and therefore a non-linear configuration. These openings are formed, firstly, by passage openings 116 for the feed hose 104, and, secondly, by the passage opening 60 for the suction line 12.

### LIST OF REFERENCE NUMERALS

2 pump

4 pump housing

6 container

8 container housing

10 reservoir

12 suction line

14 wound region

15 film

16 ventilation line

18 ventilation duct

20 inner ventilation line

22 ventilation valve

24 suction pump

25 control device

26 accumulator battery

28 inner suction line

30 exhaust

31 suction opening

32 cover element

33 suction duct

34 coupling device

36 pan

38 canister

40 locking projection

42 pawl

44 reception slot

46 rear wall

48 push button

50 integral hinge

52 actuation web

54 engagement tab

56 hinge

58 mounting segment

60 passage opening

62 tube

64 clamping cylinder

66 securing surface

68 positioning cone

70 engagement projection

72 engagement head

74 suction line port nozzle

76 ventilation line port nozzle

78 cone seat

80 ventilation interface

82 container housing wall

84 interface

88 filter

90 mounting pin mating element

92 face

94 intermediate module

96 second cover element

98 intermediate module housing

100 face

102 feed pump

104 feed hose

105 inlet and outlet openings

106 planetary gears

108 shaft

110 housing wall

112 ventilation duct segment

114 suction duct segment

116 passage opening

120 receptacle

122 membrane

124 peg

126 upper wall section

1. A device for the extraction of body fluid by suction, comprising:
a pump with a pump housing that comprises a suction opening,
a container with a container housing that surrounds a reservoir for storing the extracted body fluid and comprises an interface in a container housing wall for the communication between said suction opening and said reservoir,
a suction line port that is in communication with said reservoir and connectable to a suction line for the extraction of body fluid by suction, and
a coupling device for detachably coupling said container housing to said pump housing,
further comprising a cover element covering said coupling device.

2. The device according to claim 1, wherein said cover element is connectable to said container housing in a positive-fit and/or positive substance-fit and/or force-fit manner.

3. The device according to claim 3, said cover element comprises a push button interacting with said coupling device for decoupling said container housing from said pump housing.

4. The device according to claim 3, wherein said cover element covers a locking projection provided on said container housing or said pump housing for coupling said container housing and said pump housing and/or forms a locking projection for coupling said container housing and said pump housing.

5. The device according to claim 3, wherein said push button is arranged between two mounting segments that are formed by said cover element and connected to said container housing.

6. The device according to claim 1 further comprising, a ventilation rinsing line port for a ventilation line for supplying a fluid to the end of said suction line on the application side, where said ventilation line port is in communication with a ventilation duct leading into said pump housing.

7. The device according to claim 1 further comprising, an intermediate module which can be detachably connected by way of coupling devices to one of container housing and pump housing, which is provided with a feed pump, comprises inlet and outlet openings for a feed hose interacting with said feed pump and comprises a suction segment extending between said suction opening of said pump housing and said interface of said container housing, wherein at least one of said coupling devices is covered by said cover element.

8. The device according to claim 7 further comprising, a second cover element covering the other of said coupling devices.

9. The device according to claim 1, wherein said reservoir is provided with a filter which seals a suction duct which is in communication with said pump.

10. The device according to claim 1, wherein said cover element comprises at least one passage opening for said suction line and/or for said ventilation line and forms at least one securing surface, due to the abutment of which against said respective line, a clamping force is effected in the axial direction clamping said respective suction line against a port on the housing side.

11. The device according to claim 1, wherein said pump housing comprises at least one mounting element and said container housing at least one mounting mating element and that said container housing is pivotable relative to said pump housing by an interaction of said mounting element of said pump housing with said mounting mating element of said container housing.

12. The device according to claim 15, wherein said intermediate module comprises a mounting element and a mounting mating element and that said container housing is pivotable relative to said intermediate module by an interaction of said mounting element of said intermediate module with said mounting mating element of said container housing and that said intermediate module is pivotable relative to said pump housing by an interaction of said mounting element of said pump housing with said mounting mating element of said intermediate module.

13. The device according to claim 1 further comprising, a detection device and an identifier provided on said container housing and/or on said intermediate module, where said detection device is adapted to detect said identifier and is coupled via a signal line to a motor control device of said pump.

14. The device according to claim 1, wherein said cover element has a nonlinear arrangement of said passage openings and preferably has the shape of a segment of a circle.

15. A container for storing extracted body fluid, comprising
a container housing surrounding a reservoir for body fluid,
an interface to said reservoir connectable to a suction opening of a pump housing,
a suction line port in communication with said reservoir, and
a suction line for the extraction of body fluid by suction that is connected to said suction line port,
wherein
a cover element with at least one passage opening for said suction line and at least one securing surface, due to the abutment of which against said suction line, a clamping force is effected in the axial direction clamping said suction line against said associated suction line port.

16. The device according to claim 1, wherein that said cover element is connectable to said container housing by way of a locking mechanism.

17. The device according to claim 4, wherein said cover element covers a locking projection provided on said container housing or said pump housing for coupling said container housing and said pump housing and/or forms a locking projection for coupling said container housing and said pump housing.

18. The device according to claims 1, wherein said cover element comprises at least one passage opening for said suction line or for said ventilation line and forms at least one securing surface, due to the abutment of which against said respective line, a clamping force is effected in the axial direction clamping said respective suction line against a port on the housing side.

19. The device according to claim 1 further comprising a detection device and an identifier provided on said container housing or on said intermediate module, where said detection device is adapted to detect said identifier and is coupled via a signal line to a motor control device of said pump.
